(19) United States
(12) Patent Application Publication
(10) Pub. No.: US 2013/0053336 A1
(43) Pub. Date: Feb. 28, 2013

(54) TREATMENT OF CHLAMYDIACEAE INFECTIONS BY MEANS OF BETA-LACTAMS

(71) Applicants: Philippe Verbeke, Paris (FR); Maud Dumoux, London (GB); Colette Kanellopoulos, Versailles (FR)

(72) Inventors: Philippe Verbeke, Paris (FR); Maud Dumoux, London (GB); Colette Kanellopoulos, Versailles (FR)

(73) Assignees: UNIVERSITE PARIS DIDEROT PARIS 7; CENTRE NATIONAL DE LA RECHERCHE SCIENTIFIQUE (CNRS)

(21) Appl. No.: 13/521,909

(22) PCT Filed: Jan. 13, 2011
(86) PCT No.: PCT/EP2011/050406

§ 371 Date: Nov. 8, 2012

(30) Foreign Application Priority Data
Jan. 13, 2010 (FR) 1050203

(51) Int. Cl. A61K 31/43; A61K 31/545; A61K 31/546; A61K 31/407; A61K 31/55; A61K 31/7048; A61K 31/7052; A61P 31/04; A61P 27/02; A61P 15/00; A61P 11/00; A61P 9/00; A61P 3/06; A61P 13/02; A61P 15/08; A61P 1/16; A61P 33/02; A61P 31/10; A61P 31/12; A61P 31/22; A61P 15/02; A61K 31/424; A61K 31/431

(52) U.S. Cl. 514/30; 514/199; 514/197; 514/209; 514/207; 514/210.1; 514/196; 514/192; 514/206; 514/210.06

(57) ABSTRACT

The invention relates to a method for treating infections of bacteria of the Chlamydiaceae family using a β-lactam. The invention also relates to a method for treating diseases caused by an infection of bacteria of the Chlamydiaceae family using a β-lactam.

### INTRODUCTION

The family Chlamydiaceae comprises two genera, Chlamydia and Chlamydophila, each comprising several species; each species is composed of various biovars and serovars. Each of these species causes a set of serious pathologies in humans or animals.

Bacteria of the family Chlamydiaceae are Gram-negative. They are known as “strict intracellular” because they grow and multiply only in a parasitophorous vacuole within an infected eukaryotic cell. During development, bacteria of the family Chlamydiaceae take two forms:
the elementary body (EB), which is the infectious form and does not divide, and
the reticulate body (RB), which is noninfectious, strictly intracellular and which divides by binary fission.

The EB enters the host cell within a vesicle. If several EB infect the same cell, the vesicles assemble at the centrosome and fuse to form the inclusion. After 5 to 12 hours of infection, EB differentiate into RB, which actively multiply. After 30 to 40 hours, RB convert into EB; the inclusion and the host cell are then lysed, releasing the progeny bacteria to infect nearby cells. All these parameters can vary from one serovar and bacterial species to the next, and according to the experimental conditions (Fields, K. A. & T. Hackstadt, Annu Rev Cell Dev Biot 18:221-45, 2002).

During its development cycle, the bacterium deploys numerous strategies for surviving in the host cell. The Chlamydiaceae bacterium notably inhibits apoptosis induced by external signals, inhibits fusion of lysosomes with the bacterial inclusion, diverts vesicular traffic toward the vacuole to enable the vacuole's growth, and injects into the cytoplasm a key protease, chlamydial protease/proteasome-like activity factor (CPAF), responsible for the degradation of numerous eukaryotic proteins, including transcription factors for molecules of the major histocompatibility complex, which is responsible for presenting antigens to the immune system.

To these mechanisms developed during a continuous bacterial cycle is added the phenomenon of persistence. In response to certain stresses in the cell environment, such as the presence of certain antibiotics or of interferon gamma (IFN-γ), the bacterium stops its bacterial cycle and its proliferative mechanism, thus becoming a persistent, viable body that cannot be cultured in vitro. Persistence explains a high level of interaction between the pathogen and the host and enables the bacterium to remain alive in the organism for months. Although it is very difficult to demonstrate persistence in vivo, multiple studies suggest that a Chlamydiaceae bacterium can exist for several years within tissues such as the genital tract, conjunctival mucosa and vascular tissue, thus causing recurrent infections and chronic inflammation (Beatty et al., Microbiol Rev 58(4):686-99, 1994; Kern et al., FEMS Immunol Med Microbiol. 55(2):131-9, 2009). Chlamydia trachomatis, which is subdivided into two biovars (trachoma and lymphogranuloma) and 18 serovars (A to L), is more particularly responsible for a broad spectrum of diseases throughout the world.

For example, in developed countries, Chlamydia trachomatis serovars D to K are by far the primary cause of sexually transmitted infections of bacterial origin. The LGV biovar is thus named because it is responsible for another sexually transmitted disease, lymphogranuloma venereum, also known as Durand-Nicolas-Favre disease.

In women, genital chlamydia is frequently asymptomatic and, as a result, goes untreated. Chlamydia trachomatis can reach all levels of the genital apparatus and can cause endocervicitis of the cervix, where it constitutes a reservoir of infection.

Chlamydia trachomatis can also cause urethritis and endometritis. Chronic inflammation due to infection is the cause of total or partial obstruction of the fallopian tubes (salpingitis). Chlamydia trachomatis is the most frequent cause of typical or atypical salpingitis. Salpingitis represents the principal risk factor for tubal sterility and ectopic pregnancy in women. It should be noted that attacks on the fallopian tubes have consequences in terms of a woman's reproductive ability several months after Chlamydia trachomatis infection. There is thus a period when fertilization and passage of the fertilized egg are possible. The egg then arrives in a contaminated uterine cavity. In this case, implantation failures, growth delays in utero and premature deliveries due to inflammation of the chorion are observed.

In addition, Chlamydia trachomatis serovars A to C cause an ocular infection, trachoma, responsible for acquired blindness. The primary infection is the cause of mucopurulent conjunctivitis which is initially without sequelae. Repeated or persistent episodes of infection are the cause of chronic inflammation characterized by the presence of subepithelial follicles, followed by papillary hypertrophy of the tarsal conjunctiva of the upper eyelid. Infection of this conjunctive tissue can progress over the years, leading to inflection of the lashes toward the eye, causing irritation of the cornea (trichiasis) and thus leading to the opacification responsible for blindness.

Here still, chronic inflammation due to the presence of bacteria following reinfections or persistence is the cause of chlamydiosis sequelae. Persistence can be explained by the infection of cells in direct contact with the conjunctiva, cells of the corneal epithelium or limbal stem cells. However, since the cornea is completely avascular it is inaccessible to immune cells, which may promote persistence. With 84 million people affected (the vast majority in the world's poorest regions) including 8 million with a visual deficiency, this ancient disease was in 2007 the most preventable cause of blindness in the world. It is the object of an eradication program by the World Health Organization.

In general, Chlamydiaceae infections are treated with antibiotics capable of passing through the lipophilic plasma membrane to reach the RB, which are metabolically active and are thus most sensitive to treatment. Tetracyclines (doxycycline and tetracycline), quinolones (ofloxacin and levofloxacin) and macrolides (erythromycin and azithromycin) are the three most commonly used families of antibiotics in Chlamydiaceae infections. They are highly effective in eliminating bacteria of the family Chlamydiaceae in infected tissue, notably in the case of uncomplicated genital infections. Nevertheless, the effective cure rate is only 70% to 80%, without the patients being reinfected or the antibiotic therapy being stopped. This tends to demonstrate that the current anti-Chlamydiaceae antibiotic therapy is not completely effective. It is suggested that the ineffectiveness of these treatments among certain patients could be due, at least in part, to the presence of persistent forms of the bacterium.

The hypothesis of insensitivity of persistent forms of the bacterium to currently proposed treatments is supported by several studies carried out in vitro using bactericidal antibiotics. In these studies, only azithromycin appears effective in eradicating persistent forms of Chlamydia. Nevertheless, a study carried out in vivo demonstrated by electronic microscopy abnormal forms of the bacterium, evoking persistent bodies, in biopsies of the neck of the womb and of the male ureter in patients treated with azithromycin, which runs counter to a destroying effect of this antibiotic on persistent forms of Chlamydia (Bragina et al., J Eur Acad Dermatol Venereol. 15(5):405-9, 2001; Katz & Fortenberry, Proceedings of the Ninth International Symposium on Human Chlamydial Infection, 1998).

Vaccination is now seen as the best means of controlling Chlamydiaceae infections. Nevertheless, research into an anti-Chlamydiaceae vaccine is a complex task that requires a balance between effective protective immunity and pathology associated with this response. In addition, since persistent forms of Chlamydiaceae cannot be detected by the immune system, it is highly unlikely that a vaccine can eradicate them.

Thus, there remains a need for an effective treatment against Chlamydiaceae infections and, in particular, against persistent Chlamydiaceae infections.

The present Inventors have found in a surprising manner that persistent forms of Chlamydiaceae are sensitive to β-lactams and that β-lactams can thus be used to treat chlamydiosis.

### DESCRIPTION OF THE INVENTION

Up until now, it has been commonly accepted that β-lactams, and penicillin G in particular, induce Chlamydiaceae persistence. Treatment with penicillin G was even one of the preferred means used in the laboratory to induce persistence (Beatty et al., Microbiol. Rev. 58(4):686-699, 1994; Huston et al., BMC Microbiol. 8:190, 2006; Goellner et al., Infect Immun. 74(8):4801-8, 2006; Skilton, R. J. et al., PLoS One 4:e7723, 2009). This model was developed following observations during the 1970s that the treatment of infected cells with penicillin G affects RB division and leads to the production of an apparently persistent bacterial phenotype (Matsumoto, A. and G.P. Manire, J Bacteriol 101:278-285, 1970; Kramer, M. J. and F. B. Gordon, Infect Immun 3:333-341, 1971; How, S. J. et al., J Antimicrob Chemother 15:399-404, 1985; Kuo, C. C. et al., Antimicrob Agents Chemother 12:80-83, 1977; Huston, W. M. et al., BMC Microbiol 8:190, 2008; Skilton, R. J. et al., PLoS One 4:e7723, 2009; Johnson, F. W. and D. Hobson, J Antimicrob Chemother 3:49-56, 1977; Lambden, P. R. et al., Microbiology 152:2573-2578, 2006). However, the same studies often also showed that treatment with a β-lactam did not affect the growth kinetics of the bacterial inclusion, but led to the formation of abnormal bacteria that are much larger (5 to 10 μm) than “classic” persistent bacteria. Moreover, other studies have shown that the elimination of penicillin G does not reestablish infectivity (Johnson, F. W. and D. Hobson, J Antimicrob Chemother 3:49-56, 1977; Wolf, K. et al., Infect Immun 68:2379-2385, 2000; Peters, J. et al., Cell Microbial 7:1099-1108, 2005). Although such characteristics are inconsistent with the definition of persistence, it has nevertheless been postulated that antibiotic therapy with β-lactams could lead to persistence in bacteria in vivo. As a result, this antibiotic therapy using β-lactams was not recommended.

Consistent with a role of penicillin G in inducing persistence in Chlamydia trachomatis, the clinical tests that were performed did not demonstrate β-lactam therapeutic activity. The authors even concluded that penicillin G and other β-lactams have no effect on Chlamydia trachomatis (Heinonen et al., Genitourin Med 62:235-239, 1986; Ridgway G. L., J Antimicrob Chemother 40(3):311-4, 1997). This is the commonly accepted view today.

However, the present Inventors have shown for the first time that penicillin G, far from inducing Chlamydiaceae persistence, in fact leads to its degradation. In particular, bacteria of the family Chlamydiaceae treated with penicillin G lose any infectivity, even after the antibiotic has been removed from the medium. This therapeutic effect is not limited to penicillin G, but can be observed with at least one group of β-lactams. Said therapeutic effect is not restricted to a particular bacterial species, but can be obtained with any bacterium of the family Chlamydiaceae.

The invention thus relates to a method for treating with a β-lactam an infection with a bacterium of the family Chlamydiaceae. The invention also relates to a method for treating with a β-lactam a pathology caused by infection with a bacterium of the family Chlamydiaceae. According to a preferred aspect of the invention, said bacterium is a bacterium of the species Chlamydia trachomatis, Chlamydophila pneumoniae or Chlamydophila psittaci. According to a particularly preferred aspect of the invention, said bacterium is a bacterium of the species Chlamydia trachomatis.

According to another aspect, the invention relates to the use of a β-lactam to manufacture a drug to treat infections by a bacterium of the family Chlamydiaceae. According to still another aspect, the invention relates to the use of a β-lactam to manufacture a drug to treat pathologies caused by infection with a bacterium of the family Chlamydiaceae. According to a preferred aspect of the invention, said bacterium is a bacterium of the species Chlamydia trachomatis, Chlamydophila pneumoniae or Chlamydophila psittaci. According to a particularly preferred aspect of the invention, said bacterium is a bacterium of the species Chlamydia trachomatis.

The invention also relates to a β-lactam for use in treating infections by a bacterium of the family Chlamydiaceae. The invention also relates to a β-lactam for use in treating pathologies caused by infection with a bacterium of the family Chlamydiaceae. According to a preferred aspect of the invention, said bacterium is a bacterium of the species Chlamydia trachomatis, Chlamydophila pneumoniae or Chlamydophila psittaci. According to a particularly preferred aspect of the invention, said bacterium is a bacterium of the species Chlamydia trachomatis.

In the context of the invention, “β-lactam” refers to any antibiotic containing a β-lactam ring in its molecular structure. The β-lactams of the invention thus comprise penicillin derivatives as well as cephalosporins, monobactams, carbapenems and ⊕-lactamase inhibitors. In particular, the β-lactam of the invention can be benzylpenicillin (penicillin G), phenoxymethylpenicillin (penicillin V), ampicillin (penicillin A), benzathine benzylpenicillin, methicillin, dicloxacillin, flucloxacillin, co-amoxiclav (amoxicillin clavulanic acid), piperacillin, ticarcillin, azlocillin, carbenicillin, cephalexin, cefalotin, cefazolin, cefaclor, cefuroxime, cefamandole, cefotetan, cloxacillin, cefadroxil, cefixime, cefoxitin, ceftriaxone, cefotaxime, ceftazidime, cefepime, cefpirome, imipenem, imipenem in combination with cilastatin, cefixime in combination with imipenem, meropenem, mecillinam, ertapenem, aztreonam, clavulanic acid, tazobactam or sulbactam. In a preferred aspect of the invention, said β-lactam is not amoxicillin. According to a more preferred aspect of the invention, said β-lactam is selected from the group comprised of amoxicillin, benzylpenicillin (penicillin G), phenoxymethylpenicillin (penicillin V), cloxacillin, cefadroxil, cefixime, imipenem, cefixime in combination with imipenem, mecillinam, clavulanic acid, tazobactam and sulbactam. In an even more preferred aspect, said β-lactam is penicillin G.

Whereas the prior art teaches that penicillin G is a factor that triggers persistence, the present Inventors have shown that treatment with penicillin G leads to irreversible loss of bacterial infectivity. In particular, treatment with penicillin G leads to the degradation of Chlamydiaceae. When infected cells are treated with penicillin G, the expression of bacterial 16S ribosomal RNA (rRNA) is not detected and infected cells become sensitive to external apoptotic stimuli.

According to a more particular aspect, the invention thus relates to the use of penicillin G to manufacture a drug to treat infections by a bacterium of the family Chlamydiaceae or to treat pathologies caused by infection with a bacterium of the family Chlamydiaceae, characterized in that said treatment leads to irreversible loss of infectivity in said bacterium. This infectivity can be measured by all suitable methods at the disposal of the person skilled in the art. These methods have already been described in the prior art (see, for example, Verbeke, P. et al. PLoS Pathog 2:e45, 2006), and thus it is not necessary to detail said methods herein; an example of one such method is provided in the experimental examples.

In addition, treatment with penicillin G of the invention leads to bacterial degradation via fusion with lysosomes. Another more particular aspect of the invention thus relates to the use of penicillin G to manufacture a drug to treat infections by a bacterium of the family Chlamydiaceae or to treat pathologies caused by infection with a bacterium of the family Chlamydiaceae, characterized in that said treatment leads to degradation of said bacterium. According to an even more particular aspect of the invention, degradation of said bacterium results from fusion of the bacterium with lysosomes.

The inventors have shown that penicillin G is capable of inducing degradation of bacteria of the family Chlamydiaceae. In the context of the invention, “family Chlamydiaceae” refers to the taxonomic unit composed of two genera, Chlamydia and Chlamydophila, as defined in Bush & Everett, Int J Syst Evol Microbiol. 51(Pt 1):203-20, 2001. Similarly, in the context of the invention, “genus Chlamydia” refers to the taxonomic unit comprising the species Chlamydia trachomatis, Chlamydia suis and Chlamydia muridarum, and “genus Chlamydophila” refers to the taxonomic unit comprising the species Chlamydophila abortus, Chlamydophila psittaci, Chlamydophila caviae, Chlamydophila pecorum, Chlamydophila felis and Chlamydophila pneumoniae (Bush & Everett, Int J Syst Evol Microbiol. 51(Pt 1):203-20, 2001). It is well-known to the person skilled in the art that these species have distinct host spectra: for example, Chlamydia trachomatis and Chlamydophila pneumoniae are responsible for infections in humans, Chlamydia suis in swine and Chlamydophila abortus in ruminants. The invention is not restricted to a specific bacterial species, but can be applied to any species of the family Chlamydiaceae and, in particular, to those mentioned above.

More specifically, the Inventors have shown that penicillin G is capable of inducing degradation of Chlamydia trachomatis, regardless of its biovar or even its serovar. In the context of the invention, “Chlamydia trachomatis” refers to bacteria of the trachoma biovar as well as those of lymphogranuloma venereum biovar. Similarly, the invention is thus not restricted to a particular serovar, but can be used to treat infections by any of the 18 known serovars (A to L). In particular, the invention can be used to treat infections by one of serovars A to C as well as serovars D to K and L1 to L3.

The expressions “infection with a bacterium of the family Chlamydiaceae” and “Chlamydiaceae infection” refer to the presence in at least one cell of the body of a patient or animal of said bacterium of the family Chlamydiaceae. The infection can be genital, ocular or pulmonary; it can also affect other sites, such as the endothelium or the joints. Said bacterium can be present in EB or RB form; it can also be present in persistent form. In a more particular aspect of the invention, the bacterium is present in persistent form in the patient's body.

It is known that infections by bacteria of the family Chlamydiaceae do not necessarily lead to the appearance of symptoms. For example, it is estimated that most cervicovaginal infections are asymptomatic or result only in the appearance of minor symptoms (Paavonen and Eggert-Kruse, Hum Reprod Update 5(5): 433-47, 1999). Nevertheless, the presence of Chlamydiaceae bacteria can be detected by all means well-known to the person skilled in the art, said means requiring no explanation herein. It is sufficient to mention in particular that the use of diagnostic tests based on nucleic acid amplification, such as the polymerase chain reaction (PCR), the real-time polymerase chain reaction (RT-PCR) and the ligase chain reaction (LCR), are able to distinguish with no ambiguity people infected with a bacterium of the family Chlamydiaceae from those people not infected. In addition, measurement of 16S and 23S ribosomal RNA expression establishes that the bacterium is active metabolically and is thus viable. These tests for studying Chlamydiaceae infections are commonly used in the laboratory and will thus not be detailed herein (Mpiga and Ravaoarinoro, Microbiol Res. 161(1):9-19, 2006).

The present invention thus relates in a particular aspect to a method for treating with a β-lactam a Chlamydiaceae infection or a pathology caused by a Chlamydiaceae infection, comprising a step in which Chlamydiaceae is detected. More particularly, said detection step comprises amplification of a bacterial nucleic acid by PCR, LCR or RT-PCR.

It is well-known to the person skilled in the art that bacterial species of the family Chlamydiaceae have distinct host spectra. Similarly, the person skilled in the art knows which organisms will be infected with each of said species: it is known, for example, that Chlamydia trachomatis and Chlamydophila pneumoniae are responsible for infections in humans, Chlamydia suis in swine and Chlamydophila abortus in ruminants. The invention can thus be applied to infections caused by Chlamydiaceae in humans as well as in animals. Similarly, it is possible according to the invention to treat with a β-lactam Chlamydiaceae infections in humans as well as in animals.

Although a large number of infections caused by Chlamydiaceae are asymptomatic, said infections can nevertheless cause serious pathologies in patients or in animals. In the context of the invention, the expression “pathology caused by a Chlamydiaceae infection” refers to any pathology directly or indirectly triggered by infection with a bacterium of the family Chlamydiaceae.

In the context of the invention, pathologies caused by Chlamydiaceae infection include pathologies caused in animals by Chlamydia suis, Chlamydia muridarum, Chlamydophila abortus, Chlamydophila psittaci, Chlamydophila caviae, Chlamydophila pecorum and Chlamydophila felis. In the context of the invention, said pathologies caused by Chlamydiaceae infection thus include more particularly miscarriages and neonatal deaths caused by Chlamydophila abortus, as well as conjunctivitis, keratoconjunctivitis, purulent rhinitis, enteritis, bronchopneumonia and pneumonia caused in swine by Chlamydia suis.

In humans, these pathologies can be in particular genital pathologies or ocular pathologies, but they can also be respiratory pathologies. Thus, it is known that infections by Chlamydophila pneumoniae cause a form of pneumonia and that Chlamydophila psittaci is responsible for psittacosis. It is also known that infections by bacteria of the family Chlamydiaceae spread in the organism, thus causing numerous atypical infections and pathologies such as cardiovascular and circulatory dysfunctions (atheroma). Among the cardiovascular and circulatory dysfunctions caused by bacteria of the family Chlamydiaceae, mention may be made of valvar stenosis and atheroma, respectively. In addition, said bacteria of the family Chlamydiaceae can also cause severe arthritis.

However, in a preferred aspect of the invention, the pathologies caused by infection with Chlamydia are ocular pathologies or genital pathologies. According to a more particularly preferred aspect of the invention, the ocular pathologies caused by infections by Chlamydia include trachoma. According to another more particularly preferred aspect, the genital pathologies caused by Chlamydia include lymphogranuloma venereum (Durand-Nicolas-Favre disease). According to another more particularly preferred aspect of the invention, the human genital pathologies caused by Chlamydia include pathologies such as urethritis and orchiepididymitis, which can lead to decreased male fertility. According to still another more particularly preferred aspect of the invention, the infections by Chlamydia in women cause genital pathologies such as cervicovaginitis, cervicitis, endocervicitis, urethritis, endometritis or perihepatitis, which can give rise to complications such as upper genital tract infections and, in particular, salpingitis, which is capable of developing into, among other things, formation of pyosalpinx and hydrosalpinx, which totally obstruct the fallopian tubes. Salpingitis is thus the primary risk factor for tubal sterility and ectopic pregnancy in women.

Whereas doxycycline only provides partial improvement in salpingitis, treatment with penicillin G of the invention prevents the appearance of hydrosalpinx caused by genital Chlamydia infection. Treatment with penicillin G of the invention is capable of preventing tissue damage due to Chlamydia infection. Said treatment with penicillin G of the invention thus demonstrates greater effectiveness compared to treatment with doxycycline, which is currently the standard treatment in humans.

Genital pathologies can be caused by Chlamydia infection alone; however, they can also be caused by the combination of a Chlamydia infection and an infection with another infectious organism. In particular, the infectious organisms likely to cause said pathologies are protozoa with Trichomonas vaginalis; fungi with Candida albicans; proliferation of anaerobic bacteria (Bacteroides, Peptostreptococcus, Gardnerella vaginalis, Mobiluncus) during bacterial vaginitis, for example; other bacteria such as Haemophilus ducreyi, Mycoplasma hominis, Streptococcus, Escherichia coli, Staphylococcus, Neisseria gonorrhoeae; and viral infections, for example, with the herpes simplex virus. According to a more particular aspect of the invention, the infectious organism capable of causing genital pathology as described above is Neisseria gonorrhoeae. According to another more particular aspect of the invention, the infectious organism capable of causing genital pathology as described above is Trichomonas vaginalis.

The present invention thus also relates to a method for treating with a β-lactam a genital pathology caused by infection with a bacterium of the family Chlamydiaceae, characterized in that said β-lactam is administered with another therapeutic agent. According to a particular aspect of the invention, said another therapeutic agent is selected from agents for treating infections by Neisseria gonorrhoeae. According to another particular aspect of the invention, said another therapeutic agent is selected from agents for treating infections by Trichomonas vaginalis. Said another therapeutic agent is in particular selected from the group comprised of ceftriaxone, cefixime, spiramycin, spectinomycin, azithromycin, ofloxacin, ciprofloxacin, metronidazole, tinidazole and nimorazole.

The invention also relates to pharmaceutical compositions containing a β-lactam as an active principle. Said β-lactam can be used alone or combined with another therapeutic agent as defined above.

These compositions can be administered by oral, rectal or parenteral route or locally by topical application on the skin and mucosae, but the preferred route of administration is oral.

They can be solid or liquid and provided in dosage forms commonly used in human medicine, such as, for example, tablets, coated or uncoated, gelatin capsules, granules, suppositories, injectable preparations, eye lotions, pomades, creams and gels, which are prepared according to standard methods. The active principles can be incorporated with excipients commonly used in these pharmaceutical compositions, such as talc, gum arabic, lactose, starch, magnesium stearate, cocoa butter, aqueous or non-aqueous vehicles, animal or plant fats, paraffin derivatives, glycols, various wetting agents, dispersants and emulsifiers, and preservatives. These compositions can also be provided in powder form intended to be dissolved just before use in a suitable vehicle, for example sterile non-pyrogenic water.

The dose administered is variable according to the affection treated, the subject in question, the route of administration and the compound considered. It can be, for example, between 50 μg and 300 mg per day orally in adults.

### FIGURE LEGENDS

FIG. 1. Treatment with penicillin G does not stop the development of inclusions containing abnormal forms of Chlamydia trachomatis.

HeLa cells were infected with Chlamydia trachomatis serovar L2 and treated with IFN-γ (100 ng/ml), penicillin G (100 U/ml) or gentamicin (25 μg/l). At various times after infection (hours post-infection (hpi)), the cells were fixed and stained with an antibody against Chlamydia sp. (green in the original figure) and with Hoechst (blue in the original figure). The infected HeLa cells treated with penicillin G have abnormal bodies that grow and that are phenotypically quite different from those formed during treatment with IFN-γ. The bodies formed with penicillin G are appreciably different from “classic” persistent bodies.

Scale: 10 μm. Each experiment was reproduced at least three times.

FIG. 2. Treatment with penicillin G leads to irreversible loss of infectivity in Chlamydia trachomatis.

HeLa cells were infected with the Chlamydia trachomatis L2 serovar at 1 inclusion forming unit (IFU). At 3 hpi, 3 equal batches were distinguished and penicillin G was added at a concentration of 100 U/ml to the “penicillin G” batch (black bar) and “reversion” (gray bar) but not in the control batch (white bar). At 24 hpi, penicillin G was removed from the culture medium in the reversion group. At 48 hpi or 100 hpi, the supernatants and the cells were combined and stored at −80° C. Fresh cultures of HeLa cells were then incubated for 1 hour with cell extracts (left) or supernatants (right), incubated with cycloheximide and fixed 24 hours later. The nuclei and the inclusions were stained and counted to calculate the number of IFU/ml. Under the control conditions, significant infectious ability was detected, whereas no infectious ability could be shown after treatment with penicillin G, regardless of whether or not there was a reversion phase. This result is strong proof that penicillin G does not induce persistence. It is also a first indication of the lethality of Chlamydia.

*: significantly different from the other conditions (p<0.05)

▪: significantly different from 0 (p<0.05)

•: not significantly different from 0

FIG. 3. Treatment with penicillin G leads to the loss of control of Chlamydia trachomatis over host cell apoptosis.

A: HeLa cells were infected with Chlamydia trachomatis and treated or not treated with penicillin G at 3 hpi. At 21 hpi, staurosporine was added to the culture medium. The cells were fixed at 48 hpi and stained with an antibody against Chlamydia sp. (green in the original figure) and Hoechst (blue in the original figure).

B: Normal or apoptotic nuclei associated with inclusions were counted and the percentage of infected cells in apoptosis was calculated.

Scale: 10 μm. *: significantly different (p=0.01)

FIG. 4. Treatment with penicillin G leads to an absence of 16S rRNA expression.

HeLa cells were infected with the Chlamydia trachomatis L2 serovar and treated or not treated with penicillin G at 3 hpi. At 100 hpi, the cells were collected and the RNA extracted carefully to avoid DNA contamination. Half of the isolated RNA was used for reverse transcription. The RNA and cDNA were amplified by PCR with primers for 16S (bacterial) and 18S (eukaryotic) rRNA in order to study bacterial activity and to verify cDNA quality and quantity. PCR of the RNA is used to verify the absence of DNA contamination. It also indicates the absence of contaminant in the PCR. The infected cells treated with penicillin G express 18S RNA, but not 16S, which indicates that the abnormal bacterial forms are not viable.

FIG. 5. Treatment with penicillin G leads to the entry of lysosomes into abnormal inclusions.

HeLa cells were infected with Chlamydia trachomatis and either fixed and stained (A) or observed immediately (B and C) at 24 hpi.

A: Hoechst staining (blue in the original figure), anti-Chlamydia sp. (green in the original figure) and anti-cathepsin D (red in the original figure). Cathepsin D is strikingly present in the abnormal bodies. Scale: 10 μm.

B: The cells were incubated with LysoTracker probes for 30 minutes. The LysoTracker probes are particularly localized in abnormal inclusions with complex membrane networks. Close inspection shows very bright points in the abnormal inclusions, suggesting the entry of a lysosome into an abnormal inclusion. Tip of the arrow: inclusion membrane, arrow: compartment loaded with LysoTracker probes. DIC: differential interference contrast. Scale: 10 μm.

C: Videomicroscopy (time lapse) with a confocal microscope equipped with a high-speed scanner. In gray, differential interference contrast; in red (in the original figure), the LysoTracker probes. A lysosome (arrow), near an inclusion, enters this abnormal structure and appears to remain inside.

FIG. 6. The effect of penicillin G on Chlamydia is independent of host cell type, serovar, biovar or species of Chlamydia.

A monocyte/macrophage tumor cell line, THP-1, an endometrial tumor cell line, RL95-2, and a cervical tumor cell line, HeLa, infected with serovar L2 of the Chlamydia trachomatis LGV biovar, or with serovar D of the trachoma biovar, or with Chlamydia muridarum, and treated with penicillin G (3 hpi) exhibiting abnormal inclusions. In blue in the original figure; Hoechst, in green in the original figure: anti-Chlamydia sp. Image taken at 24 hpi (RL95-2/Chlamydia L2; HeLa/Chlamydia-D; HeLa/C. muridarum) or at 48 hpi (THP-1/Chlamydia-L2; THP-1/Chlamydia-D). Scale: 10 μm.

FIG. 7. Treatment with penicillin G leads to very rapid loss of control by Chlamydia trachomatis over host cell apoptosis.

HeLa cells were infected in the absence (white bar) or presence of penicillin G (100 U/ml) added at 2 hpi (light gray bar) or at 29 hpi (dark gray bar). Staurosporine was added at 31 hpi. The cells were fixed and observed as described below. The apoptotic or normal nuclei associated with inclusions were counted, and the percentages of apoptotic infected cells were determined. In 2 hours, treatment with penicillin G induces a loss of control by Chlamydia trachomatis over host cell apoptosis.

*: significantly different (p<0.05)

FIG. 8. Low penicillin G concentrations induce the same non-infectious state of Chlamydia trachomatis.

HeLa cells were infected with Chlamydia trachomatis serovar L2 at 1 IFU. At 3 hpi, penicillin G was added at a concentration of 1, 10 or 100 U/ml in the penicillin G (−) and reversion (+) samples. At 24 hpi, penicillin G was removed from the culture medium in the reversion group (+). At 48 hpi or 100 hpi, the supernatants and the cells were collected and stored at −80° C. HeLa cells grown on slides were incubated for 1 hour with cell extracts (left) or with supernatants (right), treated with cycloheximide and fixed 24 hours later. The nuclei and inclusions were stained and counted to determine the number of IFU/ml. Under the control conditions (progeny bacteria from untreated cells), significant infectivity was detected, whereas no reinfection was obtained with bacteria collected from cells treated in a permanent or transitory fashion with penicillin G.

*: significantly different from the other conditions (p<0.05)

▪M: significantly different from 0 (p<0.05)

•: not significantly different from 0

FIG. 9. Cathepsin D is localized in the abnormal forms of Chlamydia trachomatis independently of the biovar and the host cell type.

The cells were infected and treated at 3 hpi with penicillin G. At 24 hpi, the cells were fixed and stained with anti-Chlamydia sp. antibodies (green in the original figure) and anti-cathepsin D antibodies (red in the original figure), as well as with Hoechst (blue in the original figure).

FIG. 10. Effect of the treatment with penicillin G on the development of hydrosalpinx following infection of C57B1/6 mice with Chlamydia muridarum.

The mice were infected by vaginal route with 10⁷ IFU of Chlamydia muridarum. After ten days, one group of mice did not receive antibiotic (untreated), another was treated with 5 mg/ml doxycycline orally and the last group received 5 mg/ml penicillin G orally. The antibiotic treatment was stopped after 20 days and the mice were kept isolated for an additional 60 days. The mice were then sacrificed, the appearance of the genital tract analyzed and the presence of hydrosalpinx (arrows) sought.

### EXPERIMENTAL EXAMPLES

1. Materials

1.1. Antibodies and Reagents

Culture media (DMEM, Ham's F-12, Ham's F-12K, RPMI-1640), FCS and gentamicin solution were obtained from Invitrogen (Carlsbad, Calif., USA).

Recombinant human IFN-γ, cycloheximide, 3-methyl-adenine (3-MA), staurosporine and penicillin G were obtained from Sigma-Aldrich (St. Louis, Mo., USA). The LysoTracker probes also come from Invitrogen (Carlsbad, Calif., USA). A mixture of two FITC-conjugated mouse monoclonal antibodies against the genus Chlamydia was obtained from Argene Biosoft (Varilhes, France). Rabbit polyclonal antibodies (IgG) against cathepsin D and goat anti-rabbit antibodies conjugated to Texas Red come from Santa Cruz Biotechnology (Santa Cruz, Calif., USA). Control rabbit IgG were purified from rabbit serum using protein A-sepharose.

1.2. Cell Cultures and Bacterial Strains

All cell types (HeLa, RL-95.2, THP-1) were cultivated according to recommendations from the American Type Culture Collection (ATCC; Manassas, Va., USA), in 75 cm² culture flasks for maintenance and in covered 12- or 24-well plates or in Lab-Tek™ culture chambers for the experiments. Differentiation of THP-1 cells into macrophages was achieved using 0.25 μM PMA in the culture medium.

In order to examine whether the effects of penicillin G on bacterial infectivity depend on the strain or the serovar, ATCC serovar L2 and C. trachomatis serovar D, graciously provided by Dr. de Barbeyrac (University of Bordeaux II, Bordeaux, France) were used. C. muridarum was obtained from Dr. Roger Rank (University of Arkansas, Little Rock, Ark., USA). The bacteria were propagated as a matter of routine in HeLa cells as described above and stored at −80° C. before use (Scidmore, M. A. Curr Protoc Microbiol Chapter 1: Unit 11A 11, 2005). The number of inclusion forming units (IFU) was determined by immunofluorescence, as described above (Verbeke, P. et al. PLoS Pathog 2:e45, 2006).

2. Methods

2.1. Infection and Treatment with Penicillin G

To examine the effects of penicillin G on cell infection with Chlamydia, the host cells were cultivated up to 70% confluence without antibiotics, and then were infected with C. trachomatis strain L2 or D, or with C. muridarum at 1 IFU. The infections were carried out at 37° C. for 90 minutes with centrifugation, followed by a washing of extracellular bacteria. At 3 hpi, after the washings, the culture medium was replaced with medium containing either 25 μg/ml gentamicin or 1-100 IU/ml penicillin G. At regular intervals after treatment with penicillin G, the cultures were either fixed in 4% neutral buffered PFA for 30 minutes to be prepared for confocal microscopy, or collected to determine the infectivity of the progeny bacteria, according to the previously described method (Verbeke, P. et al. PLoS Pathog 2:e45, 2006).

2.2. Persistence Induced by Treatment with IFN-γ

HeLa cells cultured in monolayers on slides were infected with serovar L2 with an IFU of 1, as described above (Verbeke, P. et al. PLoS Pathog 2:e45, 2006). Three hours after the infection, the medium was removed and replaced with culture medium containing 500 IU/ml (final concentration) recombinant human IFN-γ. The infected cells were incubated for an additional 100 hours in the presence of IFN-γ before being prepared for confocal microscopy as described below.

2.3. Reactivation Test

HeLa cells cultured in monolayers on slides were infected with Chlamydia trachomatis serovar L2 and treated with penicillin G, as described above. Twenty hours after infection, the culture was washed and the medium replaced with medium free of penicillin G. Between 48 and 100 hpi, the cells and the culture medium were collected at various intervals and a titration test was performed according to the method of the prior art (Scidmore, M. A. Curr Protoc Microbiol Chapter 11: Unit 11A 11, 2005). To determine whether treatment with penicillin G could affect bacterial persistence, HeLa cells infected with L2 and treated with IFN-γ and penicillin G (500 IU/ml) from 48 hpi (i.e. 24 hours after treatment with IFN-γ) to 72 hpi (i.e. 48 hours after treatment with IFN-γ). The slides were then fixed and observed using an epifluorescence microscope (Leica D M, Leica, Mannheim, Germany) equipped with two sets of detection filters (Hoechst 360/40 nmBP-425 nmLP; FITC 480/40 nmBP-527/30 nmBP) and connected to a CCD camera CCC.

2.4. Sensitivity to Apoptosis

The effect of treatment with penicillin G on the anti-apoptotic activity of Chlamydia was determined. HeLa cells cultured in monolayers on slides were infected with serovar L2 and treated with penicillin G at 3 hpi or 29 hpi. At 25 hpi or 32 hpi, respectively, the cells were incubated with 1 μM staurosporine, fixed at 40 hpi or 48 hpi and prepared for epifluorescence microscopy. The percentages of infected cells containing apoptotic nuclei were calculated.

2.5. Confocal Microscopy

HeLa cells cultured in monolayers on slides were infected with serovar L2. At 3 hpi, the cells were treated or not treated with penicillin G. Next, the cells were fixed at 24 hpi, permeabilized and incubated (2 hours, room temperature) with an anti-cathepsin D antibody (1/50) in PBS/1% BSA. After several washings, a goat anti-rabbit IgG conjugated to Texas Red (1/200) was added to the slides (1 hour, room temperature). Next, the cells were counterstained for 1 hour with an FITC-conjugated anti-Chlamydia antibody (1/800) and for 5 minutes with Hoechst (1/2000). The slides were then mounted and observed using a confocal microscope (TCS Spy AOBS Tandem, Leica, Mannheim, Germany) equipped with two diode lasers (1 and 25 mW) emitting at 405 nm and 561 nm, respectively, and an argon laser (100 mW) emitting at 488 nm. The emitted signals were collected at 411-481 nm for Hoechst, 493-555 nm for FITC and 591-703 nm for Texas Red. Each experiment was performed, acquired and analyzed in a similar manner, and was repeated three times.

2.6. Labeling of Cell Cultures with LysoTracker Red.

Cells growing in Lab-Tek™ culture chambers (Thermo Fisher Scientific, Roskilde, Denmark) were infected and treated or not treated with penicillin G at 3 hpi. At 22 hpi, the cells were incubated with 10 pM LysoTracker Red diluted in DMEM (30 minutes, 37° C.). The slides were observed at 37° C. under 5% CO₂ by videomicroscopy (time lapse) using a Leica confocal microscope equipped with a tandem scanner. Excitation was at 561 nm with a laser diode (1 mW) and the emitted signal was collected between 565 and 641 nm. Images were acquired every 2 minutes for periods up to 4 hours.

2.7. RNA Preparation and Gene Expression Analysis

Roughly 1×10⁵ HeLa cells were infected and exposed or not exposed to penicillin G, as described above. At 100 hpi, total RNA were isolated using the RNeasy Plus Mini Kit (Qiagen, Hilden, Germany). The samples were treated with DNase I for 1 hour at 37° C. The enzyme was then denatured at 70° C. for 10 min. The RNA were reverse-transcribed into cDNA using random hexamer primers and AffinityScript Multiple Temperature Reverse Transcriptase (Agilent, Santa Clara, Calif., USA) and by following the manufacturer's instructions for RT-PCR. cDNA and RNA were amplified by PCR with Tag Polymerase (Invitrogen, Carlsbad, Calif., USA) in order to verify the absence of DNA contamination.

The primers used to amplify 16S rRNA are:

SEQ ID NO: 1
CGCCTGAGGAGTACACTCGC
SEQ ID NO: 2
CCAACACCTCACGGCACGAC

The primers used to amplify eukaryotic 18S rRNA are:

SEQ ID NO: 3
ATGGCCGTTCTTAGTTGGTG
SEQ ID NO: 4
CGCTGAGCCAGTCAGTGTAG

A semi-quantitative analysis of the PCR products was carried out by agarose gel electrophoresis and UV detection.

2.8. Statistical Analysis

The data are presented as a mean ± standard deviation of “n” experiments. The means ± standard deviations are shown in the figures; p-values are calculated using a paired Student's t-test. A p-value of less than 0.05 is considered statistically significant.

2.9. In Vivo Model

C57B1/6 mice were infected with Chlamydia muridarum, a Chlamydia species that infects mice and guinea pigs and is genetically close to Chlamydia trachomatis. Estrous cycles in the mice were initiated by injection of progesterone, and then the mice were infected vaginally after 3 days (10⁷ IFU). Ten days after infection, the mice were divided into three groups, a negative control group not receiving any antibiotic, a positive control group receiving doxycycline (5 mg/ml) and a test group receiving penicillin G (5 mg/ml). The duration of the treatment was 20 days; the antibiotics, doxycycline and penicillin G, were added to the mice's drinking water. After treatment, the mice were kept isolated for 60 days in order for persistent bacteria to reinitiate their cycle and for sequelae to develop. The mice were then sacrificed, the appearance of the genital tract analyzed and the presence of hydrosalpinx sought. Various organs were also taken.

3. Results

3.1. Treatment with penicillin G of cells infected with Chlamydia trachomatis serovar L2 leads to the formation of abnormal inclusions that are not persistent.

Work of the prior art portrayed the treatment of host cells in culture with penicillin G as a model of Chlamydia persistence. However, the particular characteristics of the bacterial form caused by treatment with this antibiotic, and the controversy surrounding obtaining an infectious progeny after removing penicillin G from the medium, led the Inventors to reexamine this question.

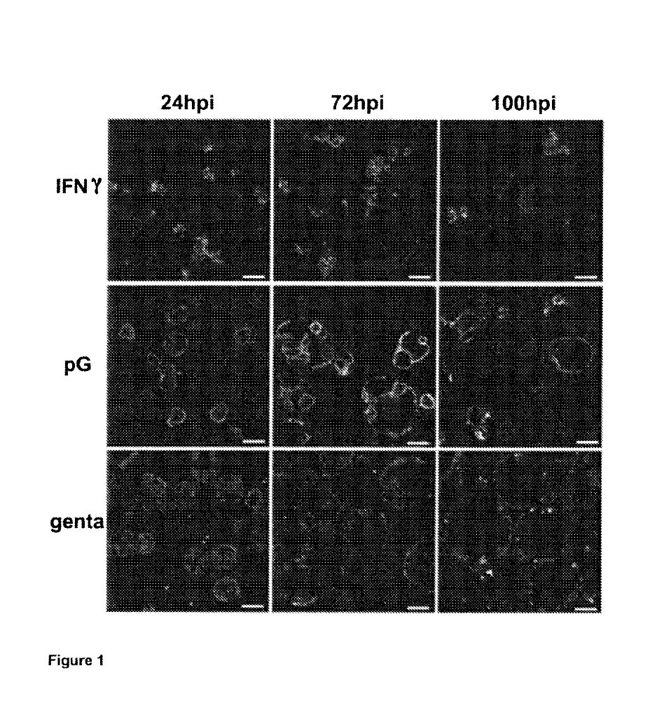

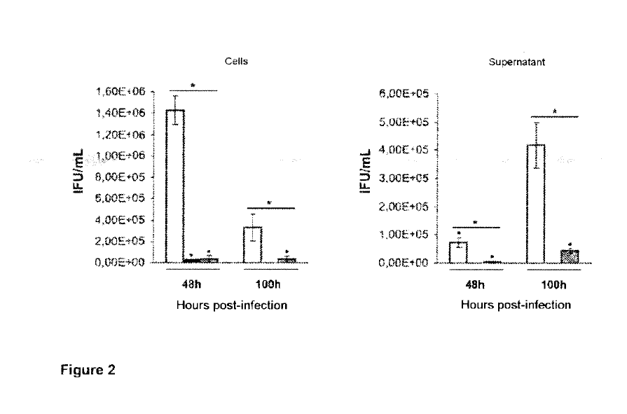

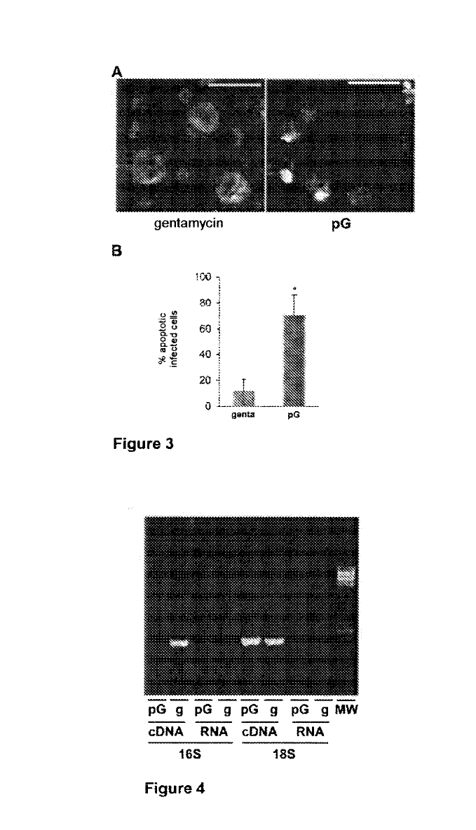

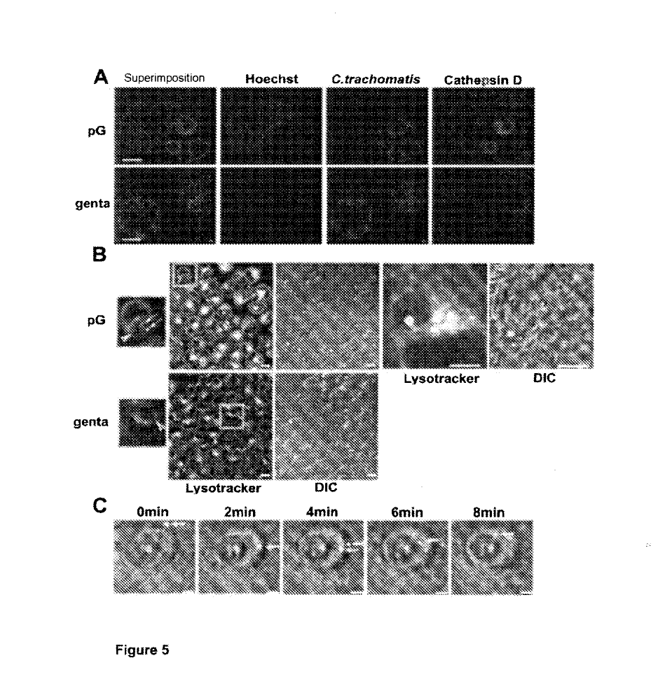

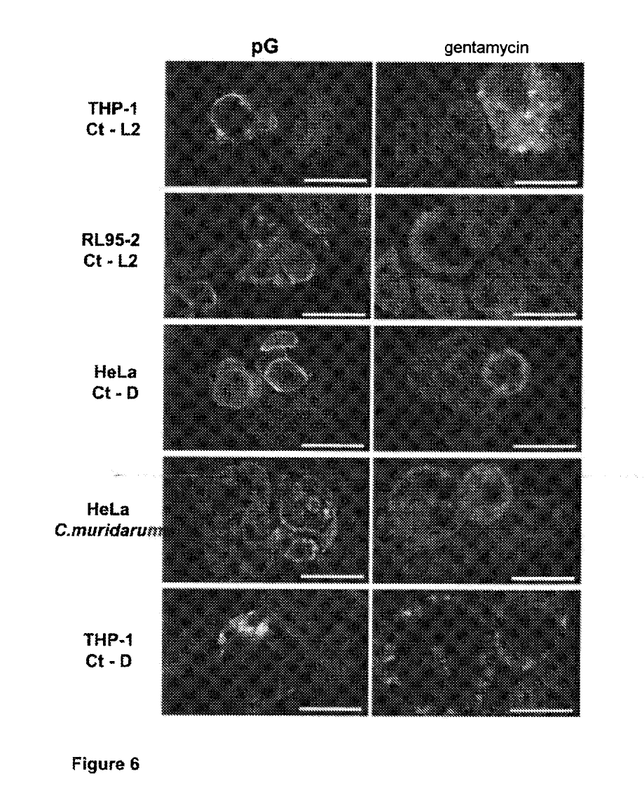

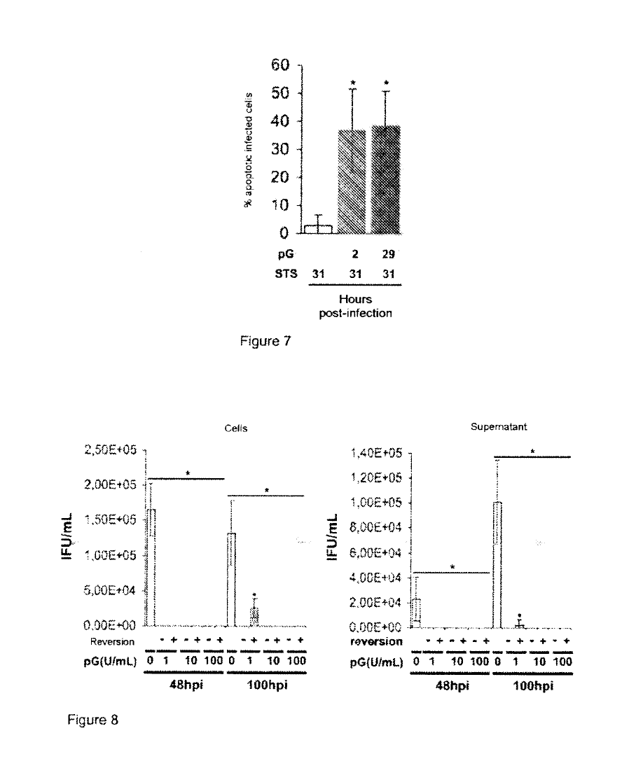

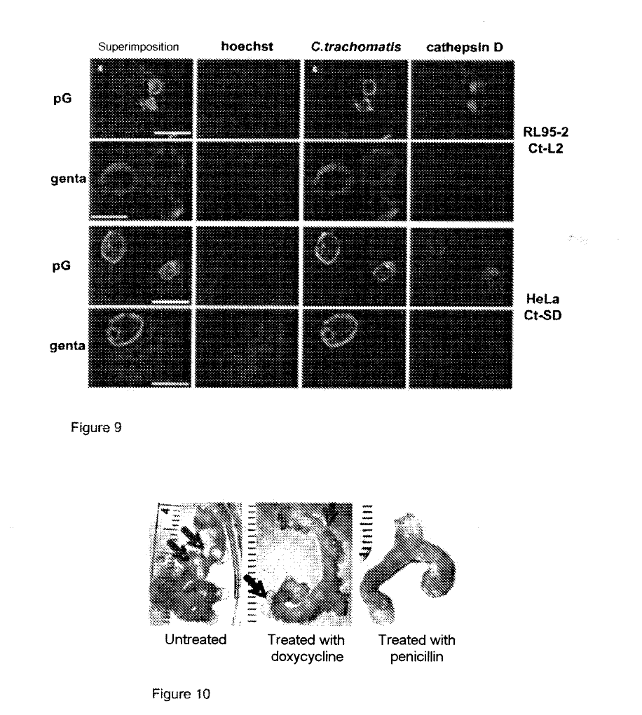

Chlamydia-infected cells were treated with gentamicin, penicillin G or IFN-γ, the latter being a well-known inducer of persistence. The size of the bacterial inclusions produced by treatment with IFN-γ remains constant when the cells are cultured for 4 days (FIG. 1). This absence of growth was observed during more than two weeks. The average diameter of the persistent forms in said inclusions was 2 to 3 μm. These two characteristics (inclusions that don't grow and that are of moderate size) are classic for

Chlamydia persistence. On the other hand, it was observed by videomicroscopy that inclusions of infected cells treated with penicillin G grow as quickly as those of cells treated with gentamicin. At 120 hours post-infection (hpi), the infected cells treated with penicillin G were lysed, releasing their structures into the culture medium. The diameter of these structures (5 to 10 μm) present in the inclusions was much greater than that of the persistent bodies obtained by treatment with IFN-γ (2 to 3 μm).

In a second series of experiments, it was shown that the generation and development of such inclusions containing abnormal bacterial particles were independent of the type of host cell, serovar, biovar or even species of Chlamydia bacterium used (FIG. 6). Moreover, infected cells were treated at various times after infection with penicillin G. It was observed that penicillin G leads to the appearance of abnormally dilated structures in inclusions only when penicillin G is added in the first phase of the bacterial cycle (before 24 hpi). This shows that penicillin G affects RB development and not EB development. As a whole, these results show that treatment with penicillin G leads to the formation of dilated bacterial forms in a non-persistent inclusion.

3.2. Treatment of host cells with penicillin G causes irreversible loss of bacterial infectivity.

When the cells were treated continuously with 100 IU/ml penicillin G, the bacteria collected at 48 hpi were several hundred times less infectious than bacteria collected from cells treated with gentamicin (FIG. 2). Under the same conditions, at 100 hpi, the bacteria released by host cells treated with penicillin G were completely noninfectious.

According to another characteristic of persistence, the inclusion can grow anew when the persistence stimulus is removed from the medium. At the same time, the persistent body is converted into a reticulate body (RB).

It was thus examined whether infectious bacteria could be recovered by removing penicillin G at 24 hpi (or 21 hours after the start of treatment with penicillin G). In this experiment, no normal development cycle in the infected cells was observed by videomicroscopy. Moreover, no improvement in infectivity was demonstrated (FIG. 2). The same irreversible loss of infectivity was observed using penicillin G concentrations up to 1 IU/ml (FIG. 7). These results show that the temporary or continuous incubation of cells infected with penicillin G totally abolishes Chlamydia infectivity.

3.3. Treatment with Penicillin G Makes Infected Cells Susceptible to Apoptosis.

The ability to control and inhibit host cell apoptosis induced by external stimuli is a major property of the Chlamydia cycle. The ability of infected cells treated with penicillin G to inhibit apoptosis triggering pathways was thus tested.

The results show that when infected cells are treated with 4 μM staurosporine at 21 hpi, only 11.83% exhibit features typical of apoptotic cells at 48 hpi, whereas uninfected control cells are all in apoptosis (FIG. 3). On the other hand, under the same conditions, 70.39% of the infected cells treated with penicillin G showed signs of apoptosis. To evaluate the speed of the penicillin G effect on loss of resistance to apoptosis, infected cells were treated with penicillin G at either 2 hpi or 29 hpi. The treated cells were then incubated with staurosporine at 31 hpi. The cells were observed 8 hours later (FIG. 8). Roughly 38% of the cells were apoptotic under both conditions, which thus indicates that penicillin G has a very rapid effect. This result shows that bacteria treated with penicillin G very quickly lose control over host cell apoptosis initiation.

3.4. Treatment with Penicillin G Leads to the Loss of 16S Ribosomal RNA Expression.

It is known that both the reticulate body and the persistent form of Chlamydia express 16S ribosomal RNA (rRNA), which is essential for the bacterium. The expression of 16S rRNA was studied in abnormal bacterial forms induced by penicillin G. At 100 hpi, the RNA were extracted from infected cells treated with penicillin G, gentamicin or IFN-γ, and 16S rRNA expression was studied by RT-PCR. A specific sequence of 16S rRNA was thus amplified from infected cells treated with gentamicin (FIG. 4) or with IFN-γ. On the other hand, infected cells treated with penicillin G do not produce bacterial RNA. However, all the infected cells expressed the same levels of eukaryotic 18S rRNA, indicating that the RNA extraction and the RT-PCR were efficient. Moreover, no DNA contamination was observed by PCR of extracted RNA. As a whole, these results show that the RB and the persistent form of Chlamydia produce 16S rRNA. On the other hand, bacteria treated with penicillin G lost the ability to express this essential RNA.

3.5. Treatment of Infected Cells with Penicillin G Leads to the Fusion of Lysosomes with the Abnormal Inclusion.

The abnormal phenotype of the bacteria treated with penicillin G, coupled with the loss of control over apoptotic functions in the host cell and with the absence of the expression of essential bacterial genes, suggests that penicillin G leads to Chlamydia degradation. The intracellular particles confined within vesicles are sent toward the lysosome to be broken down. The possibility that abnormal inclusions fuse with the lysosome was thus studied.

Cathepsins are proteases with an important role in the degradation of proteins by lysosomes. In particular, cathepsin D is a lysosomal aspartic protease. The localization of cathepsin D was studied in infected cells treated with IFN-γ and penicillin G or not treated. A 24 hpi, the majority of the abnormal bacterial forms contained cathepsin D (FIG. 5A). As expected, this marker was excluded from normal and persistent inclusions. In experiments using a control isotype, no marking was observed. These results were confirmed by the LysoTracker staining study of infected cells treated or not treated with penicillin G (FIG. 5B). LysoTracker is a probe used to detect acidic compartments such as lysosomes. The LysoTracker mechanism of retention uses its protonation, which leads to its localization in organelle membranes (Haugland, R. P. The Handbook, A guide to fluorescent probes and labeling technologies, 10th edition 2005. Invitrogen Corp', Spence M. T. Z. ed. pp 580-588). The labeling of cells infected with this probe revealed a complex tubular network in the abnormal inclusions and only in the abnormal inclusions. Moreover, points of very high intensity were observed in most of these abnormal inclusions (FIG. 5B, framed). These points could be lysosomes penetrating an abnormal inclusion. In order to test this hypothesis, the behavior of lysosomes in infected cells was studied by confocal videomicroscopy. It was thus demonstrated that compartments stained with LysoTracker probes enter abnormal inclusions (FIG. 5C). Moreover, the abnormal inclusions produced in RL95-2 cells infected with serovar L2 or in HeLa cells infected with serovar D also contain cathepsin D (FIG. 9). As a whole, these results show that penicillin G induces the fusion of lysosomes with abnormal Chlamydia inclusions.

3.6. Treatment of Persistent Bacteria with Penicillin G Induces the Fusion of Chlamydia Inclusions with Lysosomes and the Destruction of their Progeny.

The effects of penicillin G were tested on persistent infections. Cells were infected and then treated with IFN-γat 3 hpi. Once the appearance of persistent inclusions was observed under the microscope, penicillin G was added to the infected cells cultured in the presence of IFN-γ. Rapid reinitiation of inclusion growth was then observed, as well as an abnormal expansion of bacterial particles in said inclusions. Twenty-four hours after the addition of penicillin G, most of the abnormal bacterial forms contained cathepsin D. Finally, the cells were lysed 70 hours after the start of treatment with penicillin G, lysis being accompanied by the release of noninfectious bacteria. This result shows that treatment with penicillin G leads to the degradation of persistent forms of Chlamydia.

3.7. Treatment of Mice Infected with Penicillin G Leads to an Absence of Hydrosalpinx.

The effects of penicillin G were tested in a murine model of genital infection. In this model, hydrosalpinx formation in the uterine horns is observed. These inflammatory cysts, quite frequently observed in mammals in the context of pelvic infections with Chlamydia trachomatis result from the joining of walls and the collection of liquid.

Whereas the untreated mice (negative control) have very large hydrosalpinx over the length of the uterine horn, infected mice treated with doxycycline (positive control) have small hydrosalpinx in the oviduct (FIG. 10). In a striking manner, the genital tract of mice treated with penicillin G had a normal appearance, suggesting that treatment with penicillin G was capable of preventing tissue damage due to Chlamydia infection. These data are the first that show greater effectiveness by the penicillin G treatment compared to the doxycycline treatment, which is currently the standard treatment in humans.

3.8. Treatment with Other β-lactams of Cells Infected with Chlamydia trachomatis serovar L2 Leads to the Same Abnormal Forms and the Same Loss of Infectivity as Treatment with Penicillin G.

It was first sought to verify whether other molecules of the β-lactam family could induce in vitro the same abnormal and noninfectious forms of Chlamydia trachomatis as that produced in the presence of penicillin G. Titration experiments were performed on the infectious ability of bacteria from infected cells treated with different β-lactams. Cytotoxicity and minimum bactericidal concentration (MBC) were determined for each of these molecules. It was also verified whether all of these β-lactams have bactericidal action on persistent forms of Chlamydia trachomatis produced in vitro under the effect of IFN-γ. The results of these experiments are presented in Table 1 below.

TABLE 1

| Compound: | Ref. (Sigma) | Cytotox. | MBC | |
|---|---|---|---|---|
| Amoxicillin | 31586 | 0 | 0.3 | μM |
| trihydrate | | | (>0.1 | μM) |
| Penicillin G | | 0 | 0.3 | μM |
| | | | (>0.1 | μM) |
| Penicillin V | 46616 | 0 | 0.3 | μM |
| potassium salt | | | (>0.1 | μM) |
| Cloxacillin sodium | 46140 | 0 | 3 | μM |
| salt hydrate | | | (>1 | μM) |
| Cefadroxil | C7020 | 0 | 10 | μM |
| | | | (>3 | μM) |
| Cefixime trihydrate | 18588 | >400 μM | 300 | μM |
| (Ytterbium (III) | | | (>100 | μM) |
| ionophore I) | | | | |
| Imipenem | I0160 | 0 | 300 | μM |
| monohydrate | | | (>100 | μM) |
| Cefixime + Imipenem | | 0 | >>>(30 μM + | |
| | | | 30 μM) | |
| Mecillinam | 33447 | 0 | 3 | μM |
| | | | (>1 | μM) |
| Tazobactam sodium | T2820 | 0 | 30 | μM |
| salt | | | (>10 | μM) |
| Potassium | 33454 | >50 μM | 1 | μM |
| clavulanate | | | (>0.3 | μM) |
| Sulbactam sodium | Y0000529 | 0 | 10 | μM |
| | (Council of | | (>3 | μM) |
| | Europe) | | | |

In conclusion, all of the β-lactams produce the same effects as penicillin G.

1-22. (canceled)

23. A method for treating an infection by a bacterium of the family Chlamydiaceae comprising the step of administering a β-lactam to a subject in need thereof.

24. The method of claim 23, wherein said β-lactam is not amoxicillin.

25. The method of claim 23, wherein the β-lactam is selected from the group consisting of amoxicillin, benzylpenicillin (penicillin G), phenoxymethylpenicillin (penicillin V), cloxacillin, cefadroxil, cefixime, imipenem, cefixime in combination with imipenem, mecillinam, clavulanic acid, tazobactam and sulbactam.

26. The method of claim 23, wherein the β-lactam is penicillin G.

27. the method of claim 23, wherein said bacterium is in persistent form.

28. The method of claim 23, wherein the treatment leads to irreversible loss of infectivity in said bacterium.

29. The method of claim 23, wherein the treatment leads to degradation of said bacterium.

30. The method of claim 23, wherein said treatment leads to fusion of the lysosome with said bacterium.

31. The method of claim 23, wherein said bacterium of the family Chlamydiaceae is a bacterium of a species chosen among Chlamydia trachomatis, Chlamydia suis, Chlamydia muridarum, Chlamydophila abortus, Chlamydophila psittaci, Chlamydophila caviae, Chlamydophila pecorum, Chlamydophila felts and Chlamydophila pneumoniae.

32. The method of claim 23, wherein said bacterium of the family Chlamydiaceae is Chlamydia trachomatis.

33. The method of claim 23, comprising the step of further administering another therapeutic agent.

34. The method of claim 33, wherein said another therapeutic agent is a therapeutic agent for treating infections and/or pathologies caused by another infectious agent.

35. The method of claim 34, wherein said another infectious agent is selected from protozoa (notably Trichomonas vaginalis); fungi (Candida albicans); anaerobic bacteria (Bacteroides, Peptostreptococcus, Gardnerella vaginalis, Mobiluncus); other bacteria such as Haemophilus ducreyi, Mycoplasma hominis, Streptococcus, Escherichia coli, Staphylococcus, Neisseria gonorrhoeae; and viral infections, with notably herpes simplex virus.

36. The method of claim 34, wherein said another infectious agent is Neisseria gonorrhoeae or Trichomonas vaginalis.

37. The method of claim 33, wherein said another therapeutic agent is selected from the group consisting of ceftriaxone, cefixime, spiramycin, spectinomycin, azithromycin, ofloxacin, ciprofloxacin, metronidazole, tinidazole and nimorazole.

38. A method for treating a pathology caused by an infection with a bacterium of the family Chlamydiaceae comprising the step of administering a β-lactam to a subject in need thereof.

39. The method of claim 38, wherein said β-lactam is not amoxicillin.

40. The method of claim 38, wherein the β-lactam is selected from the group consisting of amoxicillin, benzylpenicillin (penicillin G), phenoxymethylpenicillin (penicillin V), cloxacillin, cefadroxil, cefixime, imipenem, cefixime in combination with imipenem, mecillinam, clavulanic acid, tazobactam and sulbactam.

41. The method of claim 38, wherein the β-lactam is penicillin G.

42. The method of claim 38, wherein said bacterium is in persistent form.

43. The method of claim 38, wherein the treatment leads to irreversible loss of infectivity in said bacterium.

44. The method of claim 38, wherein the treatment leads to degradation of said bacterium.

45. The method of claim 38, wherein said treatment leads to fusion of the lysosome with said bacterium.

46. The method of claim 38, wherein said bacterium of the family Chlamydiaceae is a bacterium of a species chosen among Chlamydia trachomatis, Chlamydia suis, Chlamydia muridarum, Chlamydophila abortus, Chlamydophila psittaci, Chlamydophila caviae, Chlamydophila pecorum, Chlamydophila felis and Chlamydophila pneumoniae.

47. The method of claim 38, wherein said bacterium of the family Chlamydiaceae is Chlamydia trachomatis.

48. The method of claim 38, wherein the pathology is ocular pathology, genital pathology, respiratory pathology, cardiovascular dysfunction or circulatory dysfunction.

49. The method of claim 48, wherein the ocular pathology is trachoma.

50. The method of claim 48, wherein the cardiovascular dysfunction is atheroma.

51. The method of claim 48, wherein the circulatory dysfunction is atheroma.

52. The method of claim 48, wherein the genital pathology is selected from the group consisting of lymphogranuloma venereum, urethritis, orchiepididymitis, cervicovaginitis, cervicitis, endocervicitis, endometritis, perihepatitis and salpingitis.

53. The method of claim 52, wherein said genital pathology is salpingitis.

54. The method of claim 38, comprising the step of further administering another therapeutic agent.

55. The method of claim 54, wherein said another therapeutic agent is a therapeutic agent for treating infections and/or pathologies caused by another infectious agent.

56. The method of claim 55, wherein said another infectious agent is selected from protozoa (notably Trichomonas vaginalis); fungi (Candida albicans); anaerobic bacteria (Bacteroides, Peplosireptococcus, Gardnerella vaginalis, Mobiluncus); other bacteria such as Haemophilus ducreyi, Mycoplasma hominis, Streptococcus, Escherichia coli, Staphylococcus, Neisseria gonorrhoeae; and viral infections, with notably herpes simplex virus.

57. The method of claim 55, wherein said another infectious agent is Neisseria gonorrhoeae or Trichomonas vaginalis.

58. The method of claim 54, wherein said another therapeutic agent is selected from the group consisting of ceftriaxone, cefixime, spiramycin, spectinomycin, azithromycin, ofloxacin, ciprofloxacin, metronidazole, tinidazole and nimorazole.
